(19) United States
(12) Patent Application Publication
(10) Pub. No.: US 2022/0380287 A1
(43) Pub. Date: Dec. 1, 2022

(54) Solids-Liquid Separation Processes

(71) Applicant: INEOS US CHEMICALS COMPANY, Naperville, IL (US)

(72) Inventor: Timothy H. Keyes, Naperville, IL (US)

(21) Appl. No.: 17/772,271

(22) PCT Filed: Oct. 30, 2020
(86) PCT No.: PCT/US2020/058348

§ 371 Date: Apr. 27, 2022

Related U.S. Application Data
(60) Provisional application No. 62/928,407, filed on Oct. 31, 2019.

(51) Int. Cl. C07C 51/47; C07C 51/265; C07C 51/43

(52) U.S. Cl. CPC C07C 51/47; C07C 51/265; C07C 51/43

(57) ABSTRACT

The present disclosure relates generally to solid/liquid separation processes. One aspect of the disclosure is a process including filtering a solid/liquid mixture comprising a solid crude aromatic carboxylic acid, a monocarboxylic acid solvent, and minor amounts of an oxidation catalyst in a feed zone of a rotary filter (e.g., a rotary pressure filter), the feed zone having at least two filter zones to form a first feed filtrate comprising monocarboxylic acid solvent and solids; and a second feed filtrate separate from the first feed filtrate, the second feed filtrate comprising monocarboxylic acid solvent and solids, the second feed filtrate being lower in solids than the first feed filtrate; and transferring at least a portion of the first feed filtrate to the reactor zone as recycle.

### RELATED APPLICATIONS

This application claims priority to U.S. Provisional Application No. 62/928,407, filed on Oct. 31, 2019, the contents of which are hereby incorporated by reference in its entirety.

### FIELD

The present disclosure relates generally to solid/liquid separations in processes for manufacturing aromatic carboxylic acids.

### TECHNICAL BACKGROUND

In processes in the chemical, food, and pharmaceutical industries, various separation techniques are used to isolate one material from another. Common techniques for separating solid materials from a liquid include vacuum or pressure filtration, drying, centrifugation, sedimentation and clarification. When a very pure solid product is required, separation may occur in multiple stages and may be combined with washing steps. For example, a solid recovered from one of the techniques noted above may be washed or reslurried with additional liquids in order to remove impurities before being subjected to another solid/liquid separation technique to recover a final, more pure product.

Multiple-stage separation techniques may result in higher purities of solid products, but may require substantially more investment in equipment. One highly successful method to reduce capital expenditures in a multi-stage separation is through the use of a rotary pressure filter apparatus. Rotary pressure filter apparatuses have been designed to perform more than one of the steps of a multiple-stage separation technique in a single piece of equipment by progressing the material being processed through separate work zones. For example, known rotary pressure filter apparatuses perform a filtration in a filter or feed zone to form a filter cake, followed by a washing of the filter cake in one or more wash zones. The washed filter cake may be dried in a drying zone before leaving the rotary pressure filter. Rotary pressure filter apparatus are generally known in the art and are disclosed, for example, in U.S. Pat. Nos. 2,741,369, 7,807,060, and U.S. Patent Application Publication No. 2005/0051473.

There remains a need to improve separation processes for aromatic carboxylic acid reaction effluents that utilize rotary pressure filter apparatus.

### SUMMARY

The scope of the present disclosure is not affected to any degree by the statements within the summary.

According to one aspect of the disclosure, a process for manufacturing an aromatic carboxylic acid includes
oxidizing in a reactor zone a feedstock comprising a substituted aromatic hydrocarbon in the presence of an oxidation catalyst and monocarboxylic acid solvent under reaction conditions suitable to form crude aromatic carboxylic acid;
cooling the reactor zone effluent to form a solid/liquid mixture comprising a solid crude aromatic carboxylic acid, a monocarboxylic acid solvent, and minor amounts of the oxidation catalyst;
filtering the solid/liquid mixture in a feed zone of a rotary filter (e.g., a rotary pressure filter), the feed zone having at least two filter zones to form
   a first feed filtrate comprising monocarboxylic acid solvent and solids, and
   a second feed filtrate separate from the first feed filtrate, the second feed filtrate comprising monocarboxylic acid solvent and solids, the second feed filtrate being lower in solids than the first feed filtrate; and
transferring at least a portion of the first feed filtrate to the reactor zone as recycle.

In certain embodiments of the processes as otherwise described herein, oxidation catalyst is recovered from the second feed filtrate in a catalyst recovery zone.

In certain embodiments of the processes as otherwise described herein, at least a portion of the effluent of the catalyst recovery zone is recycled to the reactor zone.

In certain embodiments of the processes as otherwise described herein, the ratio of the volume of the first feed filtrate to the second feed filtrate is in the range of 1:20 to 3:1.

In certain embodiments of the processes as otherwise described herein, the ratio of the volume of the first feed filtrate to the second feed filtrate is in the range of 1:5 to 2:1.

In certain embodiments of the processes as otherwise described herein, the first feed filtrate includes at least 60% of the total amount of solids of all filtrates of the filtering operation.

In certain embodiments of the processes as otherwise described herein, the solid/liquid mixture is transferred to the rotary pressure filter from a crystallization zone in which the aromatic carboxylic acid is crystallized.

In certain embodiments of the processes as otherwise described herein, the filtering operation forms a filter cake, and wherein the process further comprises washing the filter cake in a first wash zone with a first wash fluid to form a first wash filtrate.

In certain embodiments of the processes as otherwise described herein, the ratio of the volume of the first wash filtrate to the first feed filtrate is in the range of 1:2 to 5:1.

In certain embodiments of the processes as otherwise described herein, at least a portion of the first wash filtrate is transferred to the reactor zone as recycle.

In certain embodiments of the processes as otherwise described herein, at least a portion of the first feed filtrate and, optionally, at least a portion of the first wash filtrate is transferred directly to the reactor zone as recycle.

In certain embodiments of the processes as otherwise described herein, at least a portion of the first feed filtrate and, optionally, at least a portion of the first wash filtrate is transferred indirectly to the reactor zone as recycle.

In certain embodiments of the processes as otherwise described herein, at least a portion of the first feed filtrate, and optionally, at least a portion of the first wash filtrate is transferred to a drum, and then at least a portion of the filtrate is transferred from the drum to the reactor zone.

In certain embodiments of the processes as otherwise described herein, the aromatic carboxylic acid comprises terephthalic acid.

In certain embodiments of the processes as otherwise described herein, the monocarboxylic acid solvent comprises acetic acid.

Other aspects of the disclosure will be apparent to those skilled in the art in view of the description that follows.

### BRIEF DESCRIPTION OF THE DRAWINGS

FIG. 1 is a process flow diagram for the manufacture and recovery of purified forms of aromatic carboxylic acids in accordance with one embodiment of the present disclosure.

FIG. 2 shows a side view cross-section of a rotary pressure filter in accordance with one embodiment of the present disclosure.

FIG. 3 shows a front view cross-section of a rotary pressure filter shown in FIG. 2.

FIG. 4 shows a perspective view of a portion of a rotary drum of the rotary pressure filter apparatus shown in FIGS. 2-3.

FIG. 5 shows a front view cross-section of a rotary pressure filter shown in FIGS. 2-4.

### DETAILED DESCRIPTION

In various aspects, the processes of the disclosure provide an efficient manner of recovering components of the filtrate of a solid/liquid mixture filtration.

Additional features of the processes of the disclosure will now be described in reference to the drawing figures.

The present inventors have noted that the filtrate collected from the filtration can vary as the material is processed through separate work zones, complicating the treatment and/or recovery of the components of the filtrate. Specifically in the context of separation of a solid crude aromatic carboxylic acid from a monocarboxylic acid solvent (e.g., formed by an oxidation reaction in the solvent), the present inventors have noted that the filter cake of the solid crude aromatic carboxylic acid itself can provide considerable filtering functionality. However, in the initial stage of filtration of the aromatic carboxylic acid from the solvent, the filter cake has not yet set, and so some solid aromatic carboxylic acid can be included with the filtrate. This is especially true when a filter with a relatively large pore size is used. Use of a relatively large pore size is advantageous, however, for purposes of increasing flow rate and simplification of rinsing.

The present inventors have determined that a rotary pressure filter can be used to separately collect filtrates from different filter zones, such that in an initial stage of filtration in a given zone (e.g., a feed zone) the higher-in-solids first feed filtrate is collected and recycled to a reactor zone, while in a subsequent stage of filtration the lower-in-solids (or even substantially free of solids) second feed filtrate can be conducted to a catalyst recovery zone, e.g., for removal of all or a part of an oxidation catalyst. Accordingly, one aspect of the disclosure provides a process including filtering a solid/liquid mixture having a solid crude aromatic carboxylic acid and a monocarboxylic acid solvent (e.g., from a crystallization zone) in a feed zone of a rotary pressure filter having at least two filter zones to form a first feed filtrate and then a second feed filtrate separate from the first feed filtrate, each of the first feed filtrate and the second feed filtrate comprising monocarboxylic acid solvent, the second feed filtrate being lower in solids than the first feed filtrate. By “lower in solids,” it is meant that the second feed filtrate has a lower absolute amount of solids by mass than the first feed filtrate. In certain embodiments, the second feed filtrate has no more than 50%, no more than 25%, or even no more than 10% of the solids of the first feed filtrate as a mass percent. In this context, “solids” means material that is actually solid in the mixture (i.e., not dissolved solids).

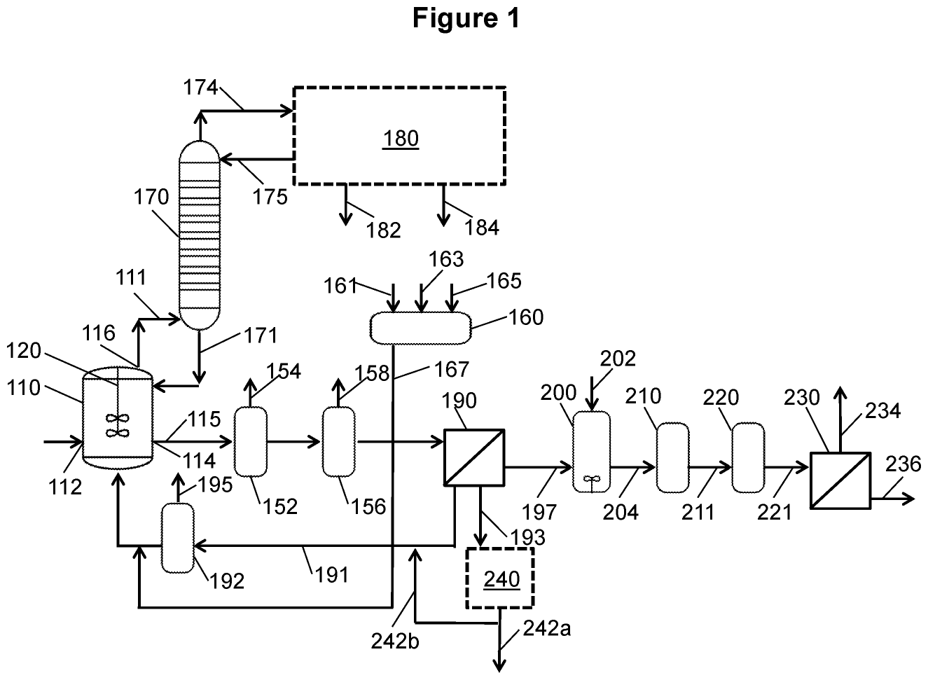

FIG. 1 is a process flow diagram for manufacturing and recovering aromatic carboxylic acids in accordance with one embodiment of the present disclosure. FIG. 1 depicts a number of additional elements for use in processes according to certain embodiments of the disclosure. A system for performing a process 100 of FIG. 1 includes a reaction zone that includes an oxidation reactor 110 configured for liquid-phase oxidation of feedstock to provide a reaction zone effluent; a crystallization zone 150 configured for forming solid crude aromatic carboxylic acid from the reaction zone effluent, and comprising crystallization vessels 152 and 156; a solid/liquid separation device 190 configured for separating solid crude aromatic carboxylic acid (and oxidation by-products) from liquid; a mixing zone including a purification reaction mixture make-up vessel 200 configured for preparing mixtures of crude aromatic carboxylic acid in purification reaction solvent; a purification zone including a hydrogenation reactor 210 configured for contacting the crude aromatic carboxylic acid with hydrogen in the presence of a catalyst to form a purified aromatic carboxylic acid; a recovery zone comprising a crystallization zone 220 including at least one crystallizer configured for forming a slurry stream comprising solid purified aromatic carboxylic acid and a vapor stream, wherein the vapor stream comprises steam and hydrogen; and a solid/liquid separation device 230 configured for separating solid purified aromatic carboxylic acid from liquid.

However, the person of ordinary skill in the art will appreciate that the integration of processes in FIG. 1 is meant to be purely representative, and various other integrated and non-integrated configurations may likewise be used.

Liquid and gaseous streams and materials used in the process represented in FIG. 1 may be directed and transferred through suitable transfer lines, conduits, and piping constructed, for example, from materials appropriate for process use and safety. It will be understood that particular elements may be physically juxtaposed and, where appropriate, may have flexible regions, rigid regions, or a combination of both. In directing streams of compounds, intervening apparatuses and/or optional treatments may be included. By way of example, pumps, valves, manifolds, gas and liquid flow meters and distributors, sampling and sensing devices, and other equipment (e.g., for monitoring, controlling, adjusting, and/or diverting pressures, flows and other operating parameters) may be present.

As described above, the feedstock includes a substituted aromatic hydrocarbon. Representative feedstock materials suitable for use in the processes of the disclosure include but are not limited to aromatic hydrocarbons substituted at one or more positions with at least one substituent that is oxidizable to a carboxylic acid group. In some embodiments, the positions of the substituents correspond to the positions of the carboxylic acid groups of the aromatic carboxylic acid being prepared. In some embodiments, the oxidizable substituents include alkyl groups (e.g., methyl, ethyl, and/or isopropyl groups). In other embodiments, the oxidizable substituents include oxygen-containing groups, such as hydroxyalkyl, formyl, aldehyde, and/or keto groups. The substituents may be the same or different. The aromatic portion of feedstock compounds may be a benzene nucleus or it may be bi- or polycyclic (e.g., a naphthalene and/or anthracene nucleus). In some embodiments, the number of oxidizable substituents on the aromatic portion of the feedstock compound is equal to the number of sites available on the aromatic portion. In other embodiments, the number of oxidizable substituents on the aromatic portion of the feedstock is fewer than all such sites (e.g., in some embodiments 1 to 4 and, in some embodiments, 2). Representative feed compounds that may be used in accordance with the present teachings—alone or in combinations—include but are not limited to toluene; ethylbenzene and other alkyl-substituted benzenes; o-xylene; p-xylene; m-xylene; tolualdehydes, toluic acids, alkyl benzyl alcohols, 1-formyl-4-methylbenzene, 1-hydroxymethyl-4-methylbenzene; methylacetophenone; 1,2,4-trimethylbenzene; 1-formyl-2,4-dimethyl-benzene; 1,2,4,5-tetramethylbenzene; alkyl-, formyl-, acyl-, and hydroxylmethyl-substituted naphthalenes (e.g., 2,6-dimethylnaphthalene, 2,6-diethylnaphthalene, 2,7-dimethylnaphthalene, 2,7-diethylnaphthalene, 2-formyl-6-methylnaphthalene, 2-acyl-6-methylnaphthalene, 2-methyl-6-ethylnaphthalene, and the like); and the like; and partially oxidized derivatives of any of the foregoing; and combinations thereof. In some embodiments, the substituted aromatic compound comprises a methyl-, ethyl-, and/or isopropyl-substituted aromatic hydrocarbon. In some embodiments, the substituted aromatic compound comprises an alkyl-substituted benzene, o-xylene, p-xylene, m-xylene, or the like, or combinations thereof.

Aromatic carboxylic acids manufactured in accordance with the present disclosure are not restricted and include but are not limited to mono- and polycarboxylated species having one or more aromatic rings. In some embodiments, the aromatic carboxylic acids are manufactured by reaction of gaseous and liquid reactants in a liquid phase system. In some embodiments, the aromatic carboxylic acid comprises only one aromatic ring. In other embodiments, the aromatic carboxylic acid comprises a plurality (e.g., two or more) of aromatic rings that, in some embodiments, are fused (e.g., naphthalene, anthracene, etc.) and, in other embodiments, are not. In some embodiments, the aromatic carboxylic acid comprises only one carboxylic acid (e.g., —CO₂H) moiety or a salt thereof (e.g., —CO₂X, where X is a cationic species including but not limited to metal cations, ammonium ions, and the like). In other embodiments, the aromatic carboxylic acid comprises a plurality (e.g., two or more) of carboxylic acid moieties or salts thereof. Representative aromatic carboxylic acids include but are not limited to terephthalic acid, trimesic acid, trimellitic acid, phthalic acid, isophthalic acid, benzoic acid, naphthalene dicarboxylic acids, and the like, and combinations thereof. In some embodiments, the present teachings are directed to manufacture of pure forms of terephthalic acid including purified terephthalic acid (PTA) and so-called medium purity terephthalic acids.

A representative type of oxidation that may be performed in the oxidation zone (e.g., oxidation reactor 110) is a liquid phase oxidation that comprises contacting oxygen gas and a feed material comprising an aromatic hydrocarbon having one or more substituents oxidizable to carboxylic acid groups in a liquid phase reaction mixture. In some embodiments, the liquid phase reaction mixture comprises a monocarboxylic acid solvent (e.g., acetic acid) and water in the presence of an oxidation catalyst comprising at least one heavy metal component (e.g., Co, Mn, V, Mo, Cr, Fe, Ni, Zi, Ce, Hf, or the like, and combinations thereof) and a promoter (e.g., halogen compounds, etc.). In some embodiments, the oxidation is conducted at elevated temperature and pressure effective to maintain a liquid phase reaction mixture and form a high temperature, high-pressure vapor phase. In some embodiments, oxidation of the aromatic feed material in the liquid phase oxidation produces aromatic carboxylic acid as well as reaction by-products, such as partial or intermediate oxidation products of the aromatic feed material and/or solvent by-products. In some embodiments, the aromatic carboxylic acid comprises terephthalic acid, and the oxidizing comprises contacting para-xylene with gaseous oxygen in a liquid phase oxidation reaction mixture that comprises acetic acid, water, and a bromine-promoted catalyst composition. The liquid-phase oxidation and associated processes may be conducted as a batch process, a continuous process, or a semi-continuous process. The oxidation may be conducted in the reaction zone, e.g., in one or more reactors.

In a representative embodiment, such as may be implemented as shown in FIG. 1, liquid feed material comprising at least about 99 wt. % substituted aromatic hydrocarbon, aqueous acetic acid solution (e.g., containing about 70 to about 95 wt. % acetic acid), soluble compounds of cobalt and manganese (e.g., such as their respective acetates) as sources of catalyst metals, bromine (e.g., hydrogen bromide) as catalyst promoter, and air, as a source of oxygen, may be continuously charged to oxidation reaction vessel 110 through inlets, such as inlet 112. In some embodiments, vessel 110 is a pressure-rated, continuous-stirred tank reactor.

In some embodiments, stirring may be provided by rotation of an agitator 120, the shaft of which is driven by an external power source (not shown). Impellers mounted on the shaft and located within the liquid body are configured to provide forces for mixing liquids and dispersing gases within the liquid body, thereby avoiding settling of solids in the lower regions of the liquid body.

In some embodiments, para-xylene is oxidized in reaction zone, predominantly to terephthalic acid. By-products that may form in addition to terephthalic acid include but are not limited to partial and intermediate oxidation products (e.g., 4-carboxybenzaldehyde, 1,4-hydroxymethyl benzoic acid, p-toluic acid, benzoic acid, and the like, and combinations thereof). Since the oxidation reaction is exothermic, heat generated by the reaction may cause boiling of the liquid phase reaction mixture and formation of an overhead gaseous stream that comprises vaporized monocarboxylic acid, water vapor, gaseous by-products from the oxidation reaction, carbon oxides, nitrogen from the air charged to the reaction, unreacted oxygen, and the like, and combinations thereof.

The gaseous stream may be removed from the reactor through vent 116 and sent in a stream 111 to a distillation column 170. The distillation column 170 is configured to separate water from the solvent monocarboxylic acid and return a monocarboxylic acid-rich liquid phase to the reactor in stream 171. A water-rich gaseous stream is removed from the distillation column 170 in stream 174 and sent for further processing to an off-gas treatment zone 180. Reflux is returned to the distillation column 170 in stream 175. The reflux liquid may include a condensed, liquid phase component 182 of the water-rich gaseous stream 174, or may include fluid from other sources, such as liquid stream 234.

The person of ordinary skill in the art will appreciate that the off-gas treatment zone can include a variety of components, for example, one or more of a condenser; a disengagement drum configured to separate the effluent of the condenser into a gas-phase component and a liquid-phase component; a scrubber configured to remove impurities (e.g., alkyl aromatic hydrocarbons, solvent monocarboxylic acid) from the gas-phase component; a catalytic oxidation (“catox”) unit configured to remove impurities (e.g., organic components, HBr) from the gas-phase component; a bromine scrubber, configured to remove bromine from the gas-phase effluent of the catox unit; and an expander and a turbine, configured to convert energy from the gas-phase component to electricity. The components of the off-gas treatment zone may be arranged in a number of configurations. For example, the gas-phase effluent of the high-pressure bromine scrubber may be sent to the expander and turbine. In another example, the gas-phase effluent of the absorber may be sent to the expander and turbine. In yet another example, the gas-phase effluent of the disengagement drum may be sent to the expander and turbine. A liquid-phase component 182 is removed from the off-gas treatment zone 180, and may include the liquid-phase effluent of the disengagement drum or the scrubber. A gas-phase component 184 is removed from the off-gas treatment zone 180, and may include the gas-phase effluent of the disengagement drum, the absorber, the high-pressure bromine scrubber, or the expander and turbine.

The person of ordinary skill in the art will appreciate that the off-gas treatment zone can be configured in a variety of manners. Examples of processing and treatment of the reaction off-gas stream, and sources of reflux fluids are more fully described in U.S. Pat. Nos. 5,723,656, 6,137,001, 7,935,844, 7,935,845, and 8,173,834.

In some embodiments, solid crude product may be recovered from the reaction zone effluent by crystallization in one or more stages, such as in a single crystallization vessel, or, as shown in FIG. 1, in a series of multiple stirred crystallization vessels. In some embodiments, the crystallization process comprises sequential reductions in temperature and pressure from earlier to later stages to increase product recover. In the embodiment shown in FIG. 1, crystallization vessels 152 and 156 may be provided in series and in fluid communication, such that product slurry from vessel 152 may be transferred to vessel 156. Cooling in the crystallizers may be accomplished by pressure release. One or more of the crystallizers may be vented, as at vents 154 and 158, to remove vapor resulting from pressure let down and generation of steam from the flashed vapor to a heat exchanger (not shown).

In certain embodiments of the processes as otherwise described herein, the aromatic carboxylic acid prepared by the process comprises terephthalic acid, and the substituted aromatic hydrocarbon of the feedstock comprises para-xylene. For example, in certain such embodiments, the substituted aromatic hydrocarbon of the feedstock is at least 99% by weight para-xylene.

A variety of process operations can be used in recovery of the crystallized carboxylic acid. In certain embodiments as otherwise described herein, at least a portion of an effluent of a last crystallizer of the crystallization zone is separated to form an aromatic carboxylic acid-rich stream and a solvent-rich stream. For example, in the process depicted in FIG. 1, the crystallization vessel 156 is in fluid communication with a solid/liquid separation device 190. The solid/liquid separation device 190 is configured to receive a slurry of solid product from the crystallization vessel 156, and is further configured to separate the slurry of solid product to form an aromatic carboxylic acid-rich stream 197 and one or more solvent-rich streams (in FIGS. 1, 191 and 193). In a representative embodiment, such as may be implemented as shown in FIG. 1, the slurry of solid product from the crystallization vessel 156 includes a solid crude aromatic carboxylic acid, a monocarboxylic acid solvent, and minor amounts of an oxidation catalyst.

Notably, the separation methods described herein can advantageously be used in the separation of at least a portion of a last crystallizer of the crystallization zone to form an aromatic carboxylic acid rich stream and one or more solvent-rich streams. Accordingly, the solid/liquid separation device 190 can be a rotary filter such as a rotary pressure filter. Suitable rotary pressure filters are sold by BHS-Sonthofen and are disclosed for example, in U.S. Pat. Nos. 2,741,369, 7,807,060, U.S. Pat. App. 20050051473, US Pat. App. 20150182890, and WO 2016/014830.

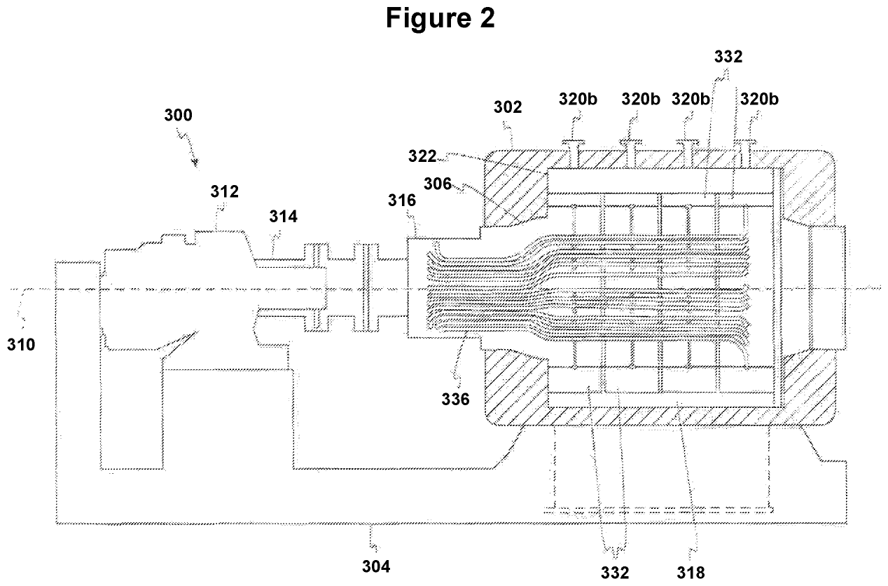

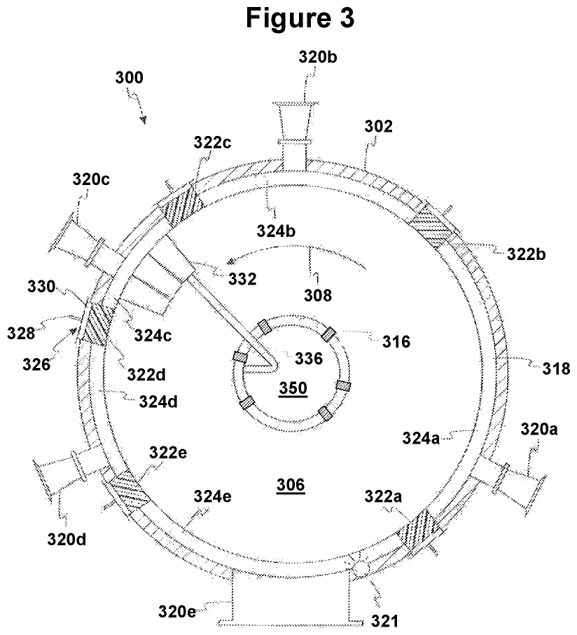

FIG. 2 is a longitudinal cross section of a rotary pressure filter apparatus in accordance with one embodiment of the present disclosure. FIG. 3 shows a front view cross-section of the rotary pressure filter shown in FIG. 2. As shown in FIG. 2, the rotary pressure filter apparatus 300 operates under a positive pressure to filter and remove liquid from a solid/liquid mixture and to collect a solid product as a final product or as an intermediate for further processing. The rotary pressure filter apparatus 300 includes a stationary housing 302 capable of withstanding an internal pressure above ambient. The housing 302 is mounted upon a frame 304. Inside the housing 302 is a rotary filter drum 306. As shown in FIG. 3, the rotary filter drum 306 rotates as indicated by arrow 308 around an axis 310 (FIG. 2) at a speed of around 0.4 to 2 RPM, and in some embodiments at a speed of about 0.8 to 1.5 RPM. The axis 310 defines a longitudinal direction of the rotary drum 306 and the rotary pressure filter apparatus 300. The rotary filter drum 306 is driven by a drive mechanism 312, which is also mounted on the frame 304. A shaft 314 connects the drive mechanism 312 to a control head portion 316 of the rotary drum 306.

The surface of the rotary drum 306 is spaced from the inside of the housing 302 such that generally annular plenum 318 is formed therebetween. Material passageways 320a, 320b, 320c, 320d, and 320e, such as inlets and outlet piping, are adapted to allow passage of material between the annular plenum 318 and a location outside the housing 302.

One or more sealing members 322a, 322b, 322c, 322d, 322e are configured to contact the rotary drum 306 and divide the annular plenum 318 into a plurality of zones 324a, 324b, 324c, 324d, 324e. The sealing members 322 generally contact the rotary drum with enough pressure to pressure seal the zones 324 from each other but still allowing the rotary drum 306 to rotate. The sealing members 322 are each part of a sealing device 326 which includes an actuating mechanism adapted to members 322 in the radial direction to exert force against the rotary drum 306. In the embodiment shown, the actuating mechanism is a pneumatic device including an inlet 328 for introducing gas into a plenum 330 to exert a pressure force against the outer surface of the respective sealing member 322. Suitable pressure forces exerted by the pneumatic device include those about 0.8 to 2.0 bar above the highest pressure in any of the zones 324a-324e of the rotary pressure filter apparatus 300. Those skilled in the art will recognize that other actuating mechanisms may be substituted for the pneumatic device.

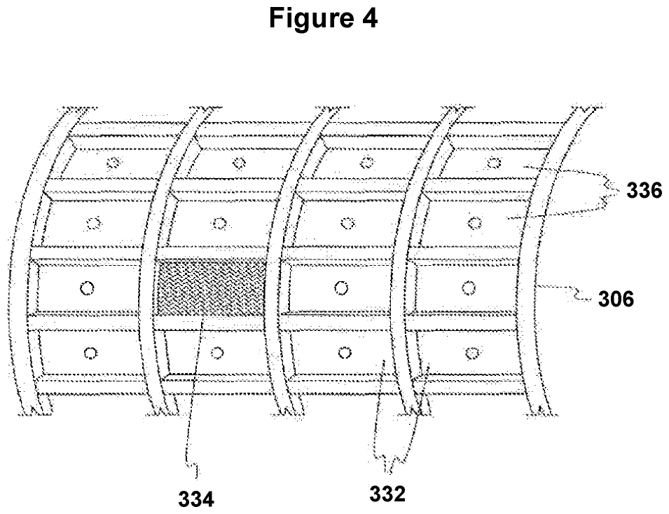

A plurality of compartments 332 are arranged around the outer surface or circumference of the rotary filter drum 306 and rotate with the filter drum 306. The compartments 332 each include a filter member 334 (shown in one compartment in FIG. 4) adjacent the filter drum. In some embodiments the filter member comprises a filter sheet supported in a filter housing (not shown), for example, by being positioned over a metal screen. In some embodiments, the filter sheet is manufactured from a polyether ketone (PEEK) polymer or a polyvinylidene difluoride (PVDF) polymer. The filter sheet can be, for example, formed as a fabric or cloth.

The present inventors have noted that it can be especially desirable to use the methods described herein in conjunction with a filter member having a relatively large pore size. While relatively large pore sizes can be desirable from the standpoint of improved flow and resistance to fouling, they can allow particulate through in the initial stage of filtration, i.e., while the filter cake is forming. The processes described herein advantageously allow for the use of a filter member with a relatively large pore size, because the first feed filtrate (relatively rich in solids particles) can be recycled to the reaction zone, with the second feed filtrate (relatively poor in solids) being further processed, e.g., by going on to a catalyst recovery zone.

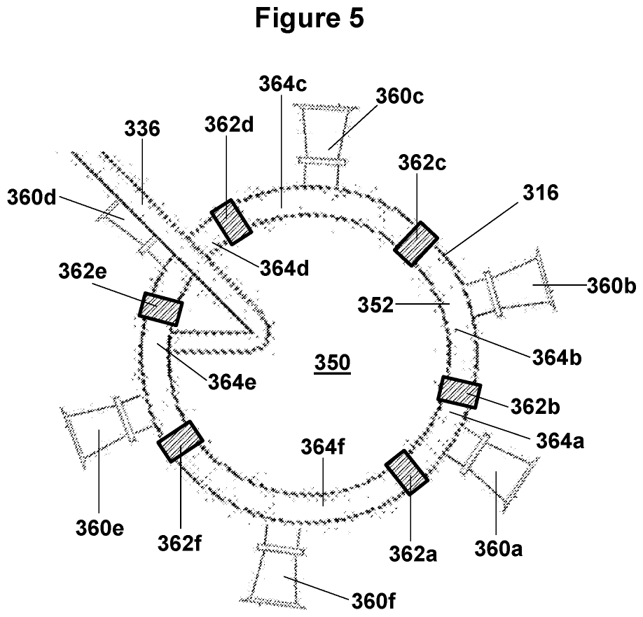

Each compartment 332 also has associated with it a corresponding outlet pipe 336 which also rotates with the filter drum 306 and the compartments 332. The outlet pipes 336 are configured such that filtrate received from each compartment 332 passes through its corresponding filter member 334 adjacent the filter drum 306 and into its corresponding outlet pipe 336. The outlet pipes 336 remove the filtrate from the compartments 332 and deliver the filtrate to the control head 316, where it is collected through one or more filter zones 364 (shown in FIG. 5), and removed from the rotary pressure filter apparatus 300.

The compartments 332 rotate with the rotary drum 306 and accordingly pass sequentially pass through each of the zones 324a, 324b, 324c, 324d, 324e. In the embodiment shown, the compartments 332 are arranged in rows of four along the longitudinal direction 310. Those skilled in the art will recognize that other configurations of the compartments would be suitable as well.

In operation, a pressurized feed containing a solid/liquid mixture is introduced into the feed inlet material passageway 320a and into plenum 318 in a first zone designated as feed zone 324a. The solid/liquid mixture is distributed into compartment 332. In some embodiments, the pressure in the feed zone is maintained at about 3 bar(g) to about 7 bar(g), and in some embodiments, 5 bar(g) to 6 bar(g). As a result of the pressure differential that is maintained between the compartments 332 and the outlet pipes 336 and across the filter member 334 in the compartments, liquid and a portion of the solid components of the solid/liquid mixture is forced through the filter member 334 into outlet pipes 336. Filtrate thus exits the rotary pressure apparatus 300 through outlet pipes 336 and is collected in a filter zone 350. A portion of the solid components of the solid/liquid mixture remains on the filter members 334 in the form of a filter cake.

As the rotary drum 306 continues into the next zone 324b, designated as a wash zone, wash fluid is introduced into plenum 318 for distribution into the compartments 332 to wash the cake remaining on the filter members 334. In some embodiments, wash fluid is introduced at a rate of about 0.5 kg to about 1.5 kg of wash fluid per 1 kg of filter cake. The wash fluid is removed, as wash filtrate, by outlet 336. In the embodiment shown, the rotary drum then continues to a second wash zone 324c, where additional wash fluid is introduced into zone 324c, designated as a second wash zone, and the cake on the filter members 334 is again washed.

The wash fluid is selected to remove impurities from the filter cake while not interfering with further processing of the filter cake to recover the final solid product. In one embodiment, the wash fluid comprises water. In another embodiment, the wash fluid comprises condensate from another portion of an integrated process.

The rotary drum 106 continues its rotation into drying zone 324d, where a hot inert drying gas is introduced in the plenum 318 to dry the filter cake on the filter members 334. As the rotary drum completes its rotation into discharge zone 324e, the dried filter cake falls from the compartments 332 by gravity into a material passageway 320e designated as a product chute (here, providing aromatic carboxylic acid-rich stream 197). A rinse solution may be injected into inlet 321 in order to clean the filter members of the compartments 332 before they continue into the next cycle through the zones.

In the embodiment shown in FIGS. 2-4, the rotary pressure filter apparatus 300 includes a single feed zone 324a, two wash zones 324b, 324c, a single dry zone 324d, and a single discharge zone 324e. In other embodiments of the invention, the rotary pressure filter apparatus could have one wash zone, or more than two, or more or fewer than a single dry zone.

The circumference of the rotary pressure apparatus 100 defines a 360° work path, with each zone 324a, 324b, 324c, 324d, and 324e defining a portion of the work path.

FIG. 5 shows a cross-sectional view of control head 316 and inner housing 350. Control head 316 is spaced from the inside of the inner housing 350 such that generally annular plenum 352 is formed therebetween. Material passageways 360a, 360b, 360c, 360d, 360e, and 360f, such as inlets and outlet piping, are adapted to allow passage of material between the annular plenum 352 and a location inside the inner housing 350. One or more sealing members 362a, 362b, 362c, 362d, 362e, and 362f are configured to contact the control head 316 and divide the annular plenum 352 into a plurality of filter zones 364a, 364b, 364c, 364d, 364e, and 364f. In the embodiment shown in FIG. 5, filter zones 364c, 364d, 364e, and 364f each comprise a portion of the work path corresponding to, respectively, first wash zone 324b, second wash zone 324c, dry zone 324d, and discharge zone 324e of the rotary filter apparatus, and filter zones 364a and 364b together comprise a portion of the work path corresponding to the feed zone 324a of the rotary pressure filter apparatus. The sealing members 362 generally contact the control head with enough pressure to pressure seal the filter zones 364 from each other but still allow the control head 316 to rotate.

Filtrate from compartments 332 is removed from the rotary pressure filter apparatus 300 through outlet pipes 336 and delivered into filter zones 364 that do not rotate with the filter drum 306. In certain embodiments, filtrate from outlet pipes 336 is collected in six filter zones, 364a, 364b, 364c, 364d, 364e, and 364f.

The circumference of the rotary pressure apparatus 300 defines a 360° work path, with each filter zone 364 associated with a portion of the work path. In the embodiment shown in FIG. 5, filtrate from compartments 332 located in a first portion of feed zone 324a will be delivered through outlet pipes 336 into filter zone 364a, and removed from the rotary pressure filter apparatus 300 through outlet piping 360a, and filtrate from compartments 332 located in a second portion of feed zone 324a will be delivered through outlet pipes 336 into filter zone 364b, and removed from the rotary pressure filter apparatus 300 through outlet piping 360b. In certain embodiments, filter zone 364a will collect filtrate from compartments 332 located in the first 40° of the feed zone 324a of rotary pressure apparatus 300 (i.e., originating at sealing member 322a, in rotation direction 308), or in the first 60°, or in the first 80°, or in the first 100°, or in the first 120°, or in the first 140°, or in the first 160°, or in the first 180°, or in the first 200°, or in the first 220° of the feed zone 324a of rotary pressure apparatus 300.

In the embodiment shown in FIG. 5, a first wash filtrate from compartments 332 located in the first wash zone 324b will be delivered through outlet pipes 336 into filter zone 364c, and removed from the rotary pressure filter apparatus 300 through outlet piping 360c.

In certain embodiments, the relative size and orientation of one or more filter zones 364 correspond to the size and orientation of one or more zones 324. For example, in the embodiment shown in FIG. 5, the size and orientation of filter zone 364c corresponds to that of first wash zone 324b, and the size and orientation of filter zone 364d corresponds to that of second wash zone 324c. In other embodiments, the relative size and orientation of filter zones 364 is selected independently of the work path of the rotary pressure apparatus 300. For example, in certain embodiments, filter zone 364a will collect filtrate from compartments 332 of the first portion of the work path of rotary pressure apparatus 300 such that about 5 vol. % of the total filtrate delivered to the annular plenum 352 is collected in filter zone 364a, or about 10 vol. %, or about 15 vol. %, or about 20 vol. %, or about 30 vol. %, or about 40 vol. %, or about 50 vol. %, or about 60 vol. % of the total filtrate delivered to the annular plenum 352 is collected in filter zone 364a.

In certain embodiments, the ratio of the volume of the first feed filtrate (collected in filter zone 364a) to the second feed filtrate (collected in filter zone 364b) is in the range of 1:20 to 3:1, or 1:10 to 3:1, or 1:5 to 3:1, or 1:20 to 2.5:1, or 1:20 to 2:1, or 1:10 to 2.5:1, or 1:5 to 2:1. In the embodiment shown in FIG. 5, the first feed filtrate collected in filter zone 364a and the second feed filtrate collected in filter zone 364b each comprise monocarboxylic acid solvent. The person of ordinary skill in the art will appreciate that the filter cake of a compartment 332 can become more compressed as the compartment 332 moves in the rotation direction 308 (e.g., through zone 324a). Accordingly, the amount of solids forced through the filter member 334 into outlet pipe 336 can decrease as the compartment 332 rotates with the rotary drum 306. Notably, then, the presently disclosed processes can provide a second feed filtrate substantially lower in solids than the first feed filtrate.

In certain desirable embodiments, the first feed filtrate includes a high proportion of the total amount of solids collected in the filtrate streams of the filtering operation. For example, in certain embodiments, the first feed filtrate includes at least 60%, at least 75%, or even at least 90% of the total amount of solids of all of the filtrates of the filtering operation.

The first feed filtrate is transferred to a reaction zone as recycle. In certain embodiments, the first feed filtrate is indirectly transferred to oxidation reactor 110 as recycle. For example, in the embodiment shown in FIG. 5, the first feed filtrate is transferred through line 191 to mother liquor drum 192, and a portion of the mother liquor is transferred from drum 192, through line 193, to oxidation reactor 110. In another example, in the embodiment shown in FIG. 5, an overhead gaseous stream of the mother liquor drum 192 (i.e., comprising the first feed filtrate) can be condensed and transferred to solvent drum 160 (e.g., through line 161) along with a condensed gaseous overhead stream of a crystallizer (e.g., from lines 154 and/or 158, through line 163) and/or make-up solvent (e.g., through line 165). In such embodiments, a portion of the solvent is transferred from drum 160, through line 167 and line 193, to oxidation reactor 110. In other embodiments, the first feed filtrate is directly transferred to a reaction zone.

In certain embodiments, the second feed filtrate is transferred through line 193 to a catalyst recovery zone 240. Notably, the second filtrate feed, having a low solids content, can be especially suitable for catalyst recovery processes. In catalyst recovery zone 240, all or a part of an oxidation catalyst can be removed from the second feed filtrate to provide a stream poor in catalyst 242. A portion of the stream poor in catalyst 242a is purged, and a portion of the stream poor in catalyst 242b is transferred to a reaction zone (here, indirectly, with solvent-rich stream 191 to mother liquor drum 192, and then to oxidation reactor 110). In a representative embodiment, such as may be implemented as shown in FIG. 1, cobalt compounds are recovered from the second feed filtrate.

In certain embodiments, the ratio of the volume of the first wash filtrate (collected in filter zone 364c) to the first feed filtrate (collected in filter zone 364a) is in the range of 1:2 to 5:1, or 1:1 to 5:1, or 2:1 to 5:1, or 3:1 to 5:1, or 1:2 to 4:1, or 1:2 to 3:1, or 1:2 to 2:1, or 1:1 to 4:1, or 1:1 to 3:1. In certain embodiments, the first wash filtrate is transferred to a reaction zone as recycle. Notably, the first wash filtrate, having a low solids content but a relatively high water content, can desirably be directed to a zone other than the catalyst recovery zone, where one or more components of the wash filtrate could interfere with catalyst recovery processes. In certain embodiments, the first wash filtrate is combined with the first feed filtrate and transferred directly to a reaction zone. In certain embodiments, the first wash filtrate is combined with the first feed filtrate and transferred indirectly to a reaction. For example, in the embodiment shown in FIG. 5, the first wash filtrate and the first feed filtrate are combined and then transferred through line 191 to mother liquor drum 192, and a portion of the mother liquor is transferred from drum 192, through line 193, to oxidation reactor 110.

As shown in FIG. 1, the aromatic carboxylic acid-rich stream 197 from the separation device 190 comprising crude solid product may be directed to a mixing zone including a reaction mixture make up vessel 200. The crude solid product in stream 197 may be mixed and slurried in make-up vessel 200 with a make-up solvent entering vessel 200 through line 202 to form a purification reaction mixture comprising crude aromatic carboxylic acid. The purification reaction mixture prepared in vessel 200 is withdrawn through line 204. In some embodiments, the purification make-up solvent contains water. In some embodiments, the solvent line 202 connects to a holding vessel (not shown) for containing make-up solvent. In other embodiments, the solvent comprises fresh demineralized water fed from a deaerator. In other embodiments, the solvent is supplied from another part of the integrated process 100. For example, in one embodiment, the solvent comprises liquid-phase component 182 of off-gas treatment zone 180. In another embodiment, the solvent comprises the liquid-phase stream 234 exiting solid/liquid separator 230. Sources of purification make-up solvent are more fully described, for example, in U.S. Pat. Nos. 5,723,656, 6,137,001, 7,935,844, 7,935,845, and 8,173,834.

In certain embodiments as otherwise described herein, at least a portion of the aromatic carboxylic acid-rich stream is purified in a purification zone comprising a hydrogenation catalyst under reaction conditions suitable to form a purification effluent comprising purified aromatic carboxylic acid. For example, in the process depicted in FIG. 1, purification reaction mixture exiting vessel 200 through stream 204 enters purification reactor 210 of the purification zone. The purification zone may further include a pump and one or more heat exchangers (not shown) configured to pre-heat the purification mixture exiting vessel 200 before it enters purification reactor 210. In some embodiments, the purification reactor 210 is a hydrogenation reactor and purification in the purification reactor 210 comprises contacting the purification reaction mixture comprising crude aromatic carboxylic acid with hydrogen in the presence of a hydrogenation catalyst. In some embodiments, at least a portion of a purification effluent comprising purified aromatic carboxylic acid may be continuously removed from hydrogenation reactor 210 in stream 211 and directed to a crystallization zone 220 downstream of the purification zone. Crystallization zone 220 may comprise a plurality of crystallizers. In some embodiments, in crystallization zone 220, purified aromatic carboxylic acid and reduced levels of impurities may be crystallized from the reaction mixture. The resulting solid/liquid mixture comprising purified carboxylic acid solids formed in crystallization zone 220 may be fed, in stream 221, to a solid/liquid separation device 230, configured to separate the solid/liquid mixture into a liquid-phase stream 234 and an aromatic carboxylic acid-rich stream 236 comprising solid purified aromatic carboxylic acid.

The entire contents of each and every patent and non-patent publication cited herein are hereby incorporated by reference, except that in the event of any inconsistent disclosure or definition from the present specification, the disclosure or definition herein shall be deemed to prevail.

The foregoing detailed description and the accompanying drawings have been provided by way of explanation and illustration, and are not intended to limit the scope of the appended claims. Many variations in the presently preferred embodiments illustrated herein will be apparent to one of ordinary skill in the art, and remain within the scope of the appended claims and their equivalents.

It is to be understood that the elements and features recited in the appended claims may be combined in different ways to produce new claims that likewise fall within the scope of the present disclosure. Thus, whereas the dependent claims appended below depend from only a single independent or dependent claim, it is to be understood that these dependent claims can, alternatively, be made to depend in the alternative from any preceding claim—whether independent or dependent—and that such new combinations are to be understood as forming a part of the present specification.

1. A process for manufacturing an aromatic carboxylic acid comprising
oxidizing in a reactor zone a feedstock comprising a substituted aromatic hydrocarbon in the presence of an oxidation catalyst and monocarboxylic acid solvent under reaction conditions suitable to form crude aromatic carboxylic acid;
cooling the reactor zone effluent to form a solid/liquid mixture comprising a solid crude aromatic carboxylic acid, a monocarboxylic acid solvent; and minor amounts of the oxidation catalyst;
filtering the solid/liquid mixture in a feed zone of a rotary pressure filter, the feed zone having at least two filter zones to form
a first feed filtrate comprising monocarboxylic acid solvent and solids; and
a second feed filtrate separate from the first feed filtrate, the second feed filtrate comprising monocarboxylic acid solvent and solids, the second feed filtrate being lower in solids than the first feed filtrate; and
transferring at least a portion of the first feed filtrate to the reactor zone as recycle.

2. The process of claim 1, further comprising recovering oxidation catalyst from the second feed filtrate in a catalyst recovery zone.

3. The process of claim 2, wherein at least a portion of the effluent of the catalyst recovery zone is recycled to the reactor zone.

4. The process of claim 1, wherein the ratio of the volume of the first feed filtrate to the second feed filtrate is in the range of 1:20 to 3:1.

5. The process of claim 1, wherein the ratio of the volume of the first feed filtrate to the second feed filtrate is in the range of 1:5 to 2:1.

6. The process of claim 1, wherein the first feed filtrate includes at least 60% of the total amount of solids of all of the filtrates of the filtering operation.

7. The process of claim 1, wherein the solid/liquid mixture is transferred to the rotary pressure filter from a crystallization zone in which the aromatic carboxylic acid is crystallized.

8. The process of claim 1, wherein the filtering operation forms a filter cake, and wherein the process further comprises washing the filter cake in a first wash zone with a first wash fluid to form a first wash filtrate.

9. The process of claim 8, wherein the ratio of the volume of the first wash filtrate to the first feed filtrate is in the range of 1:2 to 5:1.

10. The process of claim 8, further comprising transferring at least a portion of the first wash filtrate to the reactor zone as recycle.

11. The process of claim 1, comprising directly transferring at least a portion of the first feed filtrate to the reactor zone as recycle.

12. The process of claim 1, comprising indirectly transferring at least a portion of the first feed filtrate to the reactor zone as recycle.

13. The process of claim 12, comprising
transferring at least a portion of the first feed filtrate to a drum; and then
transferring at least a portion of the filtrate from the drum to the reactor zone.

14. The process of claim 1, wherein the aromatic carboxylic acid comprises terephthalic acid.

15. The process of claim 1, wherein the monocarboxylic acid solvent comprises acetic acid.

16. The process of claim 8, comprising directly transferring at least a portion of the first feed filtrate and, optionally, at least a portion of the first wash filtrate to the reactor zone as recycle.

17. The process of claim 8, comprising indirectly transferring at least a portion of the first feed filtrate and, optionally, at least a portion of the first wash filtrate to the reactor zone as recycle.

18. The process of claim 17, comprising
transferring at least a portion of the first feed filtrate and, optionally, at feast a portion of the first wash filtrate to a drum; and then
transferring at least a portion of the filtrate from the drum to the reactor zone.

19. The process of claim 14, wherein the monocarboxylic acid solvent comprises acetic acid.
